(19) United States
(12) Patent Application Publication
(10) Pub. No.: US 2025/0186420 A1
(43) Pub. Date: Jun. 12, 2025

(54) POLYMETHINIUM SALTS AS INHIBITORS OF DIHYDROOROTATE DEHYDROGENASE

(71) Applicant: UNIVERZITA KARLOVA, Praha 1 (CZ)

(72) Inventors: Michal MASARIK, Bilovice nad Svitavou (CZ); Jindriska FIALOVA, Zelenec (CZ); Jiri NEUZIL, Praha 4-Modrany (CZ); Renata ZOBALOVA, Ricany u Prahy (CZ); Tomas BRIZA, Praha 4-Kunratice (CZ); Robert KAPLANEK, Praha 10-Hostivar (CZ); Zdenek KEJIK, Sokolov (CZ); Pavel MARTASEK, Praha 9-Vinor (CZ); Milan JAKUBEK, Rychvald (CZ)

(73) Assignee: UNIVERZITA KARLOVA, Praha 1 (CZ)

(21) Appl. No.: 18/840,866

(22) PCT Filed: Feb. 21, 2023
(86) PCT No.: PCT/CZ2023/050008

§ 371 Date: Aug. 22, 2024

(30) Foreign Application Priority Data
Feb. 23, 2022 (CZ) PV 2022-86

(51) Int. Cl. A61K 31/4439; A61K 31/404; A61K 31/428; A61K 31/498; A61P 35/04

(52) U.S. Cl. CPC A61K 31/4439; A61K 31/404; A61K 31/428; A61K 31/498; A61P 35/04

(57) ABSTRACT

Derivatives of polymethinium salts of the general formula I, where both terminal heteroaromatic groups of the methinium chain are identical or different and are benzothiazole, naphthothiazole, benzimidazole, naphthoimidazole, benzooxazole, naphthooxazole, benzoselenazole, naphthoselenazole, quinoline, benzoquinoline, indole or benzoindole, the particular structure of which is characterized by the groups A, B, X, Y, with one or more R groups on both terminal heteroaromatic groups of the methinium salt, where R is H, C1 to C12 alkyl, glycol chains with 1 to 8 glycol (OCH2CH2) repeating units terminating with an O—C1 to C12 alkyl substituent or OH group, alkyl C1 to C8 sulfonic acids or their corresponding lithium, sodium or potassium salts, allyl, propargyl, phenyl, benzyl, pyridyl, halogen, CH₂OR′, OR′, CHF₂, CF₃, OCF, OCOR′, CN, CHO, COOR′, CONHR′, CONR′₂, CONHOR′, CONHNHR′, CONHNR′₂, N₃, NO₂, SR′, SCN, NHR′, NR′₂, NHCOR′, NHCONHR′, NHCONR′₂, NHCSNHR′, NHCSNR′₂, NHSO₂NHR′, NHSO₂NR′₂, NHCOOR′, N(COOR′)₂, B(OR′)₂, SO₃R′, SO₂NHR′, SO₂NR′₂, SO₂R′, where R′ is H, alkyl C1 to C12, phenyl, p-tolyl, benzyl, allyl, propargyl, CF₃; CH═CH—CH═CH (i.e. a fused benzene nucleus), where X is O, S, Se, CR′₂, NR′, CH═CH, where R′ has the above meaning; where A is alkyl C1 to C12, benzyl, allyl, propargyl, a glycol chain with a number of 1 to 8 glycolic (CH₂CH₂O) repeating units terminating with the substituent R′, (CH₂)jCOR′, (CH₂)jCOOR′, (CH₂)jSO₃R′, (CH₂)jSO₃H, (CH₂)jCONHR′, (CH₂)jCONR′₂, where j is in the range of 1 to 12 and R′ has the above meaning; where B is phenyl, pyridyl, pyrazinyl, quinolyl, quinoxalyl, furanyl, thienyl, benzoxazolyl, benzothiazolyl, which may be further substituted by one or more of the same or different substituents R, where R has the above meaning, where in the case of doubly charged salts the group B is formed by pyridyl or quinolyl quaternized on its nitrogen atom by A, where A is as defined above, where Y is acetate, acetylacetate, adipate, ascorbate, benzoate, besylate, borate, bromide, butanoate, citrate, deoxycholate, dihydrogen phosphate, phenylacetate, fluoride, phosphate, fumarate, gallate, glutarate, hexafluorophosphate, hippurate, hydrogen sulfate, chloride, perchlorate, cholate, isocyanate, isonicotinate, iodide, caprylate, cyanate, lactate, laurate, lithocholate, malate, maleate, malonate, mandelate, mesylate, monohydrogen phosphate, formate, myristate, napsylate, nicotinate, nitrate, nonafluorobutylsulfonate, oleate, oxalate, oxopropanoate, palmitate, picrate, pimelate, propionate, rhodanide, salicylate, sebacate, cinnamate, stearate, suberate, succinate, sulfate, tetrafluoroborate, tosylate, triflate, trifluoroacetate, trichloroacetate, carbonate, valerate, tartrate, and salts of natural amino acids, for the use as an inhibitor of dihydroorotate dehydrogenase.

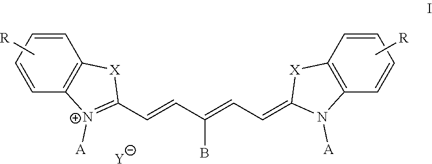

### TECHNICAL FIELD

The invention relates to the use of derivatives of polymethinium salts for the preparation of a drug for treatment using inhibition of dihydroorotate dehydrogenase.

### BACKGROUND ART

Mitochondria play a very important role in cellular metabolism. (Mori M P, Penjweini R, Knutson J R, Wang P Y, Hwang P M. Mitochondria and oxygen homeostasis. FEBS J. 2021 Jul. 8. doi: 10.1111/febs. 16115.) They provide energy in the form of ATP, regulate its distribution, and in addition they are involved in a number of physiological and pathophysiological processes in cells, such as proliferation, differentiation, information transfer and apoptosis, and play an important role in the regulation of cell growth and the cell cycle. This importance of mitochondria comes from a number of biochemical processes that take place in them. Among the most important elements of the mitochondrial metabolic pathways are the enzyme complexes of the electron transport chain and the enzyme dihydroorotate dehydrogenase (DHODH).

Electron transport chain (ETC) complexes, components of oxidative phosphorylation, are central players in mitochondrial energy production. (Caruana NJ, Stroud DA. The road to the structure of the mitochondrial respirators chain supercomplex. Biochem Soc Trans. 2020 Apr. 29; 48 (2): 621-629. doi: 10.1042/BST20190930.) The mammalian respiratory chain consists of five complexes. That is, complex I, II, III, IV and V embedded in the inner mitochondrial membrane (the arrangement is shown in FIG. 1) and mobile electron carriers, which are ubiquinone (UbQ) and cytochrome c, which together create an electron flow maintaining the potential of the mitochondrial membrane and electrochemical proton gradient leading to ATP production.

Dihydroorotate dehydrogenase (DHODH) is an enzyme involved in the de novo synthesis of pyrimidine bases. This pathway consists of six steps, with the first three steps leading to the conversion of glutamine to dihydroorotate catalyzed by a CAD polypeptide with three enzymatic activities in the cytosol. (Zhou Y, Tao L, Zhou X, Zuo Z, Gong J, Liu X, Zhou Y, Liu C, Sang N, Liu H, Zou J, Gou K, Yang X, Zhao Y. DHODH and cancer: promising prospects to be explored. Cancer Metab. 2021 May 10; 9 (1): 22. doi: 10.1186/s40170-021-00250-z.) The conversion of dihydroorotate to orotate is catalyzed by DHODH, which is found in the inner mitochondrial membrane. Orotate is converted to uridine monophosphate (UMP), again in the cytosol, which catalyzes the polypeptide uridine monophosphate synthase with two enzymatic activities. During the conversion of dihydroorotate to orotate, two electrons are released, which react in the inner mitochondrial membrane with the oxidized form of coenzyme Q, ubiquinone (UbQ). It is thus reduced to ubiquinol (UbQH). UbQH₂ subsequently transfers electrons to the UbQ molecule in mitochondrial complex III, whereby it reoxidizes itself back to UbQ and can again receive two more electrons created by the conversion of dihydroorotate to orotate. For the efficient course of de novo synthesis of pyrimidines, when UMP is the first pyrimidine and precursor for other pyrimidines, the activity of a part of the oxidative phosphorylation, which is complex III and complex IV, is essential, because complex IV accepts electrons from complex III and transfers them to molecular oxygen to form water. Complexes III and IV thus catalyze the so-called redox cycle of coenzyme Q, which directly ‘drives’ the de novo synthesis of pyrimidines via the DHODH enzyme.

Mitochondrial DHODH is a key enzyme in the de novo synthesis of pyrimidines. DHODH supplies electrons for UbQ, which is essential to maintain its functionality. When oxidative phosphorylation (OXPHOS) is dysfunctional, the UbQ redox cycle is interrupted and DHODH is inactive. In our work, we showed that DHODH activity is essential for tumor formation and that deletion of this enzyme or its inhibition suppresses tumor formation and cancer cells are arrested at the beginning of the S-phase of the cell cycle (Bajzikova M, Kovarova J, Coelho A, Boukalova S, Oh S, Rohlenova K, Svec D, Hubackova S, Endaya B, Judasova K, Bezawork-Geleta A, Kluckova K, Chatre L, Zobalova R, Novakova A, Vanova K, Ezrova Z, Maghzal G, Olsinova M, Krobova L, An Y J, Davidova E, Nahacka Z, Sobol M, Cunha-Oliveira T, Sandoval-Acuna C, Strnad H, Zhang T, Huynh T, Serafim T L, Hozak P, Sardao V A, Koopman W J H, Ricchetti M, Oliveira P J, Kolar F, Kubista M, Truksa J, Dvorakova-Hortova K, Pacak K, Gurlich R, Stocker R, Zhou Y, Berridge M V, Park S, Dong L F, Rohlena J, Neuzil J (2019) Reactivation of dihydroorotate dehydrogenase by respiration restores tumor growth of mitochondrial DNA-depleted cancer cells. Cell Metab 29, 399-416, Hubackova S, Davidova E, Boukalova S, Kovarova J, Bajzikova M, Coelho A, Terp M G, Ditzel H, Rohlena J, Neuzil J (2020) Targeting of dihydroorotate dehydrogenase and checkpoint kinase 1 results in suppression of tumor growth via cell cycle arrest induced by replication stress. Cell Death Disease 11, 110). In contrast to inhibition of DHODH, suppression of ATP OXPHOS production may not lead to suppression of tumorigenesis. Since the de novo synthesis of pyrimidines is an essential part of cell proliferation, inhibitors of DHODH activity can be expected to have a broad-spectrum antitumor effect.

Potential properties of polymethinium salts (PMS) useful in medicine, e.g. toxicity against tumor cells, mitochondrial localization and possible selectivity for tumor markers have already been described. However, their effect was not associated with DHODH inhibition. It was, for example, their application as photosensitizers (Krejcir R, Briza T, Sterba M, Simoncik O, Muller P, Coates P J, Martasek P, Vojtesek B, Zatloukalova P. Anticancer pentamethinium salt is a potent photosensitizer inducing mitochondrial disintegration and apoptosis upon red light illumination. J Photochem Photobiol B. 2020 August; 209:111939. doi: 10.1016/j.jphotobiol.2020.111939.), as apoptotic substances (Krejcir R, Krcova L, Zatloukalova P, Briza T, Coates P J, Sterba M, Muller P, Kralova J, Martasek P, Kral V, Vojtesek B. A Cyclic Pentamethinium Salt Induces Cancer Cell Cytotoxicity through Mitochondrial Disintegration and Metabolic Collapse. Int J Mol Sci. 2019 Aug. 28; 20 (17): 4208. doi: 10.3390/ijms20174208.), (Bříza T, Králová J, Dolenský B, Rimpelová S, Kejík Z, Ruml T, Hajdúch M, Džubák P, Mikula I, Martásek P, Poučková P, Král V. Striking antitumor activity of a methinium system with incorporated quinoxaline unit obtained by spontaneous cyclization. Chembiochem. 2015 Mar. 2; 16 (4): 555-8. doi: 10.1002/cbic.201402662.), inducers of the transcription factor NF-κB (Bříza T, Králová J, RIMPELOVÁ S, Havlík M, Kaplánek R, Kejík Z, Martásek P, Mikula I, Džubák P, Hajdúch M, Ruml T, Král V. Pentamethinium salts as ligands for cancer: Sulfated polysaccharide co-receptors as possible therapeutic target. Bioorg Chem. 2019 February; 82:74-85. doi: 10.1016/j.bioorg.2018.02.011.), their application as fluorescent mitochondrial probes (Bříza, S. Rimpelová, J. Králová, K. Záruba, Z. Kejík, T. Ruml, P. Martasek, V. Král, Pentamethinium fluorescent probes: The impact of molecular structure on photophysical properties and subcellular localization, Dyes and Pigments, 107, 2014, Pages 51-59, ISSN 0143-7208, https://doi.org/10.1016/j.dyepig.2013.12.021.), (Rimpelová S, Bříza T, Králová J, Záruba K, Kejík Z, Císařová I, Martásek P, Ruml T, Král V. Rational design of chemical ligands for selective mitochondrial targeting. Bioconjug Chem. 2013 Sep. 18;24 (9): 1445-54. doi: 10.1021/bc400291f.), (Král V, Bríza T, Kejík OF, Králová J, Rimpelová S, Ruml T, Martásek P. Preparation of gamma heteroaryl substituted symmetric polymethinium salts as mitochondrial probes patent CZ304094B6. 2013), (Kral V, Havlik M, Kaplanek R, Briza T, Kejik Z, Martasek P, Krcová L, Kralova J, Ruml T, Rimpelova S. Imaging agents and methods patent WO2018206126A1. 2018), (Mikšátková L, Rimpelova S, Havlík M, Dolenský B, Vellieux F, Ruml T, Martasek P, Kral V, Bríza T, Highly selective mitochondrial probes based on fluorinated pentamethinium salts: On two-photon properties and microscopic applications. Dyes and Pigments. 2020 172, 107802. doi: 10.1016/j.dyepig.2019.107802.), or targeting tumor cells based on the recognition of sulfated polysaccharides expressed by tumor cells (Bríza T, Kejík Z, Císarová I, Králová J, Martásek P, Král V. Optical sensing of sulfate by polymethinium salt receptors: colorimetric sensor for heparin. Chem Commun (Camb). 2008 Apr. 28; (16): 1901-3. doi: 10.1039/b718492a), (Král V, Králova J, Martasek P, Briza T, Kejik Z, Use of polymethine salts as sensors for tumor markers patent CZ304948B6. 2015), or the slowing down of tumor growth based on affinity of pentamethine salts for oncogenic signaling molecules, e.g. sulfated sterols (Kejík Z, Bříza T, Králová J, Mikula I, Poučková P, Martásek P, Král V. New method for recognition of sterol signaling molecules: methinium salts as receptors for sulfated steroids. Steroids. 2015 February; 94:15-20. doi: 10.1016/j.steroids.2014.10.009.).

Several DHODH inhibitors have already been described; an example is C07 (Lolli ML, Sainas S, Pippione A C, Giorgis M, Boschi D, Dosio F. Use of human Dihydroorotate Dehydrogenase (hDHODH) Inhibitors in Autoimmune Diseases and New Perspectives in Cancer Therapy. Recent Pat Anticancer Drug Discov. 2018; 13 (1): 86-105. doi: 10.2174/1574892812666171108124218.), DD264 (Lucas-Hourani M, Dauzonne D, Jorda P, Cousin G, Lupan A, Helynck O, Caignard G, Janvier G, André-Leroux G, Khiar S, Escriou N, Desprès P, Jacob Y, Munier-Lehmann H, Tangy F, Vidalain P O. Inhibition of pyrimidine biosynthesis pathway suppresses viral growth through innate immunity. PLOS Pathog. 2013; 9 (10): e1003678. doi: 10.1371/journal.ppat.1003678.), DSM265, DSM430 and DMS 450 (Phillips M A, Lotharius J, Marsh K, White J, Dayan A, White K L, Njoroge J W, El Mazouni F, Lao Y, Kokkonda S, Tomchick D R, Deng X, Laird T, Bhatia S N, March S, Ng C L, Fidock D A, Wittlin S, Lafuente-Monasterio M, Benito F J, Alonso L M, Martinez M S, Jimenez-Diaz M B, Bazaga S F, Angulo-Barturen I, Haseld en J N, Louttit J, Cui Y, Sridhar A, Zeeman A M, Kocken C, Sauerwein R, Dechering K, Avery V M, Duffy S, Delves M, Sinden R, Ruecker A, Wickham K S, Rochford R, Gahagen J, lyer L, Riccio E, Mirsalis J, Bathhurst I, Rueckle T, Ding X, Campo B, Leroy D, Rogers M J, Rathod P K, Burrows J N, Charman S A. A long-duration dihydroorotate dehydrogenase inhibitor (DSM265) for prevention and treatment of malaria. Sci Transl Med. 2015 Jul. 15;7 (296): 296ra111. doi: 10.1126/scitranslmed.aaa6645.), HZ05 (Popova G, Ladds MIGW, Johansson L, Saleh A, Larsson J, Sandberg L, Sahlberg S H, Qian W, Gullberg H, Garg N, Gustavsson A L, Haraldsson M, Lane D, Yngve U, Lain S. Optimization of Tetrahydroindazoles as Inhibitors of Human Dihydroorotate Dehydrogenase and Evaluation of Their Activity and In Vitro Metabolic Stability. J Med Chem. 2020 Apr. 23;63 (8): 3915-3934. doi: 10.1021/acs.jmedchem.9b01658.), IBC (Wu D, Wang W, Chen W, Lian F, Lang L, Huang Y, Xu Y, Zhang N, Chen Y, Liu M, Nussinov R, Cheng F, Lu W, Huang J. Pharmacological inhibition of dihydroorotate dehydrogenase induces apoptosis and differentiation in acute myeloid leukemia cells. Haematologica. 2018 September; 103 (9): 1472-1483. doi: 10.3324/haematol.2018.188185.), (Lolli M L, Sainas S, Pippione A C, Giorgis M, Boschi D, Dosio F. Use of human Dihydroorotate Dehydrogenase (hDHODH) Inhibitors in Autoimmune Diseases and New Perspectives in Cancer Therapy. Recent Pat Anticancer Drug Discov. 2018; 13 (1): 86-105. doi: 10.2174/157489281 2666171108124218), IMU838 (Fitzpatrick L R, Deml L, Hofmann C, Small J S, Groeppel M, Hamm S, Lemstra S, Leban J, Ammendola A. 4SC-101, a novel immunosuppressive drug, inhibits IL-17 and attenuates colitis in two murine models of inflammatory bowel disease. Inflamm Bowel Dis. 2010 October; 16 (10): 1763-77. doi: 10.1002/ibd.21264.), OSU-03012 (Yang C F, Gopula B, Liang J J, Li J K, Chen S Y, Lee Y L, Chen C S, Lin Y L. Novel AR-12 derivatives, P12-23 and P12-34, inhibit flavivirus replication by blocking host de novo pyrimidine biosynthesis. Emerg Microbes Infect. 2018 Nov. 21;7 (1): 187. doi: 10.1038/s41426-018-0191-1.), BAY-2402234 (Christian S, Merz C, Evans L, Gradl S, Seidel H, Friberg A, Eheim A, Lejeune P, Brzezinka K, Zimmermann K, Ferrara S, Meyer H, Lesche R, Stoeckigt D, Bauser M, Haegebarth A, Sykes D B, Scadden D T, Losman J A, Janzer A. The novel dihydroorotate dehydrogenase (DHODH) inhibitor BAY 2402234 triggers differentiation and is effective in the treatment of myeloid malignancies. Leukemia. 2019 October; 33 (10): 2403-2415. doi: 10.1038/s41375-019-0461-5. Epub 2019 Apr. 2.), PP-001 (Lolli ML, Sainas S, Pippione A C, Giorgis M, Boschi D, Dosio F. Use of human Dihydroorotate Dehydrogenase (hDHODH) Inhibitors in Autoimmune Diseases and New Perspectives in Cancer Therapy. Recent Pat Anticancer Drug Discov. 2018; 13 (1): 86-105. doi: 10.2174/1574892812666171108124218.), P1788 (Hayek S, Pietrancosta N, Hovhannisyan A A, Alves de Sousa R, Bekaddour N, Ermellino L, Tramontano E, Arnould S, Sardet C, Dairou J, Diaz O, Lotteau V, Nisole S, Melikyan G, Herbeuval J P, Vidalain P O. Cerpegin-derived furo [3,4-c]pyridine-3,4 (1H,5H)-diones enhance cellular response to interferons by de novo pyrimidine biosynthesis inhibition. Eur J Med Chem. 2020 Jan. 15; 186:111855. doi: 10.1016/j.ejmech.2019.111855.), TAK-632 (Abt E R, Rosser E W, Durst M A, Lok V, Poddar S, Le T M, Cho A, Kim W, Wei L, Song J, Capri J R, Xu S, Wu N, Slavik R, Jung M E, Damoiseaux R, Czernin J, Donahue T R, Lavie A, Radu C G. Metabolic Modifier Screen Reveals Secondary Targets of Protein Kinase Inhibitors within Nucleotides Metabolism. Cell Chem Biol. 2020 Feb. 20;27 (2): 197-205.e6. doi: 10.1016/j.chembiol.2019.10.012.), or Vidofludimus (Fitzpatrick L R, Deml L, Hofmann C, Small J S, Groeppel M, Hamm S, Lemstra S, Leban J, Ammendola A. 4SC-101, a novel immunosuppressive drug, inhibits IL-17 and attenuates colitis in two murine models of inflammatory bowel disease. Inflamm Bowel Dis. 2010 October; 16 (10): 1763-77. doi: 10.1002/ibd.21264.) Although these already published substances show high inhibitory activity for DHODH in some cases, their selectivity for the inner mitochondrial membrane, where DHODH is located, is low. For this reason, their potential therapeutic application will require a high dose of the given substance, which strongly increases the risk of side effects. In addition, these substances have a completely different structural motif than the polymethinium salts of general formula I, which are the subject of this patent. While the structural motif of polymethinium salts shows significant selectivity for the inner mitochondrial membrane. This allows for significant accumulation of the DHOH inhibitor in the inner mitochondrial membrane in close proximity to DHOH. This enables highly efficient targeting of DHODH and medicinally relevant inhibition in living systems even at very low concentrations with low risk of side effects, in contrast to known inhibitors that are not routinely tested for their intracellular distribution.

### DISCLOSURE OF INVENTION

The subject of the invention is the use of polymethinium salts of general formula I,

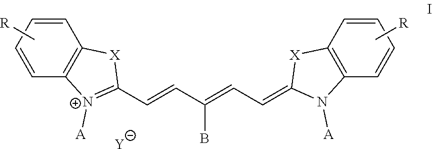

where both terminal heteroaromatic groups of the methinium chain are identical or different and are benzothiazole, naphthothiazole, benzimidazole, naphthoimidazole, benzooxazole, naphthooxazole, benzoselenazole, naphthoselenazole, quinoline, benzoquinoline, indole or benzoindole, the specific structure of which is characterized by the groups A, B, X, Y, with one or more R groups on both terminal heteroaromatic groups of the methinium salt, where R is H, C1 to C12 alkyl, glycol chains with 1 to 8 glycol (OCH₂CH₂) repeating units ending with an O—(C1 to C12) alkyl substituent or OH group, alkyl C1 to C8 sulfonic acid or their corresponding lithium, sodium or potassium salts, allyl, propargyl, phenyl, benzyl, pyridyl, halogen, CH₂OR′, OR′, CHF₂, CF₃, OCF₃, OCOR′, CN, CHO, COOR′, CONHR′, CONR′₂, CONHOR′, CONHNHR′, CONHNR′₂, N₃, NO₂, SR′, SCN, NHR′, NR′₂, NHCOR′, NHCONHR′, NHCONR′₂, NHCSNHR′, NHCSNR′₂, NHSO₂NHR′, NHSO₂NR′₂, NHCOOR′, N(COOR′)₂, B(OR′)₂, SO₃R′, SO₂NHR′, SO₂NR′₂, SO₂R′, where R′ is H, C1 to C12 alkyl, phenyl, p-tolyl, benzyl, allyl, propargyl, CF₃; CH═CH—CH═CH (i.e. a condensed benzene nucleus), where X is O, S, Se, CR′₂, NR′, CH═CH, where R′ has the above meaning; where A is a C1 to C12 alkyl, benzyl, allyl, propargyl, glycol chain with a number of 1 to 8 glycol (CH₂CH₂O) repeating units ending with the substituent R′, (CH₂)ⱼCOR′, (CH₂)ⱼCOOR′, (CH₂)ⱼSO₃R′, (CH₂)ⱼSO₃H, (CH₂)ⱼCONHR′, (CH₂)ⱼCONR′₂, where j is in the range of 1 to 12 and R′ has the above meaning; where B is phenyl, pyridyl, pyrazinyl, quinolyl, quinoxalyl, furanyl, thienyl, benzoxazolyl, benzothiazolyl, which may be further substituted by one or more of the same or different substituents R, where R has the above meaning, where in the case of doubly charged salts the group B form pyridyl or quinolyl quaternized on its nitrogen atom by group A, where A has the above meaning, where group Y is acetate, acetylacetate, adipate, ascorbate, benzoate, besylate, borate, bromide, butanoate, citrate, deoxycholate, dihydrogen phosphate, phenylacetate, fluoride, phosphate, fumarate, gallate, glutarate, hexafluorophosphate, hippurate, hydrogen sulfate, chloride, perchlorate, cholate, isocyanate, isonicotinate, iodide, caprylate, cyanate, lactate, laurate, lithocholate, malate, maleate, malonate, mandelate, mesylate, monohydrogen phosphate, formate, myristate, napsylate, nicotinate, nitrate, nonafluorobutylsulfonate, oleate, oxalate, oxopropanoate, palmitate, picrate, pimelate, propionate, rhodanide, salicylate, sebacate, cinnamate, stearate, suberate, succinate, sulfate, tetrafluoroborate, tosylate, triflate, trifluoroacetate, trichloroacetate, carbonate, valerate, tartrate, and salts of natural amino acids, for the preparation of a medicament for treatment using DHODH inhibition.

As part of our studies, we observed that these substances show a very strong inhibitory activity of mitochondrial metabolism. A detailed study of the mechanism of action surprisingly revealed that these substances are very potent and highly selective DHODH inhibitors (Example 6 and 7 and FIGS. 2 and 3). Substances 1 and 2 caused a decrease in DHODH-dependent respiration in PC-3 and U-2 OS tumor lines, while the PNT1A cell line representing a model of normal prostate tissue was unaffected by these substances. In both tumor lines, the reduction in respiration was significant (Example 6 and FIG. 2). Also, the determined binding energy values show a strong inhibitory activity of substances 1 and 2 for the given enzyme (Example 4). In addition, we observed that the effect of these substances (inhibition of enzyme activity, cytotoxicity, suppression of cell migration and invasiveness) is specific for tumor cell lines compared to non-tumor cells (Example 6 and 8-10, FIGS. 2 and 4-7). Tumor cells in the presence of these substances migrate significantly more slowly, and this phenomenon is significant for tumor lines PC-3 and U-2 OS (Example 9 and FIG. 5). The effect of substances on cell movement was verified by another method, which also enables the evaluation of cell invasiveness, which is directly related to metastatic potential (Example 10 and FIG. 6). After using the substances, the cells were not able to invade through the Matrigel layer (extracellular matrix model) and thus the affected cells at the level of the organism will not be able to intra- and extravasate (generally invade) and form secondary tumor foci. Microscopic experiments also confirm their effect on mitochondrial metabolism and vitality (Example 11 and FIG. 7). After the application of substances, mitochondrial activity is disrupted and the number of active mitochondria is reduced, which is manifested by the reduction of the mitochondrial network, which was observed by the probe staining only active mitochondria (FIG. 7A, C, D). In addition, these substances also caused mitochondria to be found only around the nucleus and not located in the leading edge of the cell, where their occurrence is essential for the ability to migrate/invade. The obtained results could not be predicted and had to be obtained experimentally.

### BRIEF DESCRIPTION OF DRAWINGS

FIG. 1 shows the structures of substances 1 (PMS1) and 2 (PMS2).

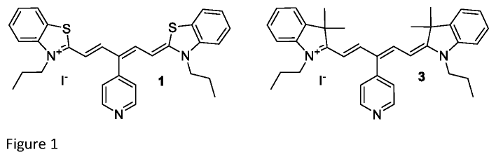

FIG. 2 shows the effect of substances 1 and 2 on DHODH inhibition. The figure depicts effect of substances 1 (top) and 2 (bottom) on relative DHODH respiration.

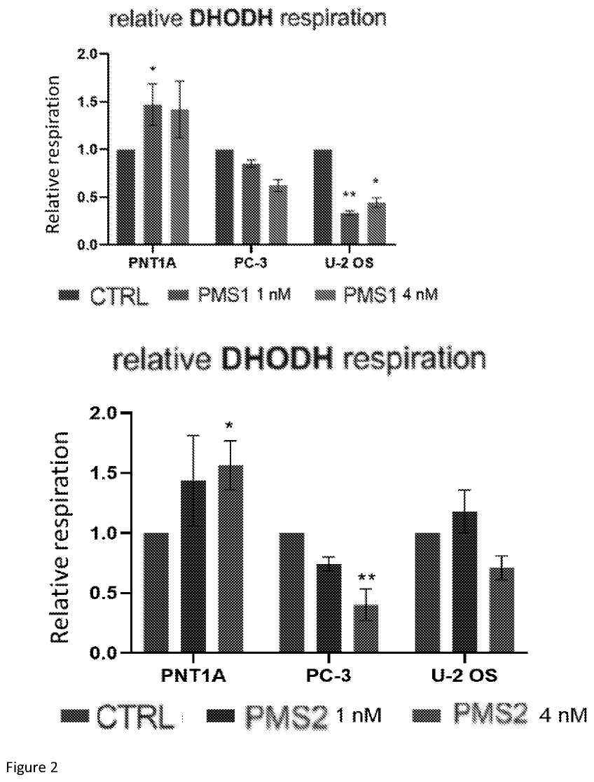

FIG. 3 shows the interactions of BAY-2402234, 1 and 2 with the 3U20 protein. The figure depicts surface view of the protein (3U20, left) with the active site (indicated in green) containing the inhibitor and docking of the inhibitor in the active site of the protein (3U20, right), blue lines show hydrogen bonds. Substance BAY-2402234 is first from the top, substance 1 is second from the top, substance 2 is third from the top.

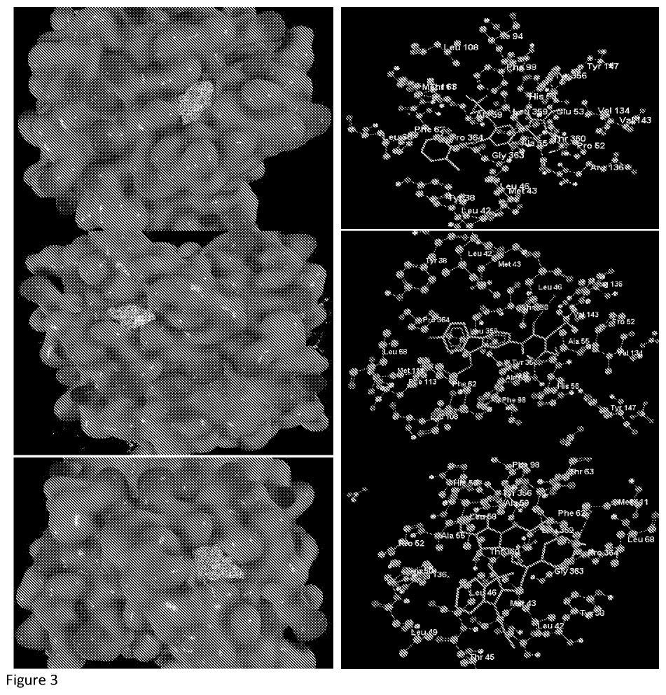

FIG. 4 shows the cytotoxicity of substances 1 and 2 against PNT1A, PC-3 and U-2 OS. The figure depicts determination of the cytotoxicity degree of substances 1, and 2, against selected cell lines (PNT1A PC-3 and U-2 OS). The values shown in the graph show the concentration needed to inhibit 50% of the cell population.

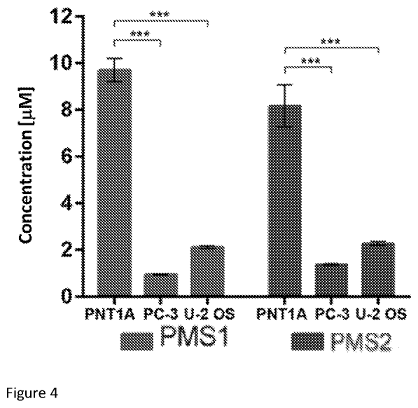

FIG. 5 shows the effect of substances 1 and 2 on cell migration using the “wound-healing assay” method. The figure depicts determination of the effect of substances 1 and 2 on cell migration by the “wound-healing assay” method for the U-2 OS cell line. PNT1A cells represent healthy prostate tissue and PC-3 and U-2 OS cell lines represent tumor tissue. A) overview graph for comparing individual experiments; B) determination of migration inhibition for substances 1 and 2 by the “wound-healing assay” method for the U-2 OS cell line; C) determination of migration inhibition for substances 1 and 2 by the “wound-healing assay” method for the PC-3 cell line; D) determination of migration inhibition for substances 1 and 2 by the “wound-healing assay” method for the PNT1A cell line.

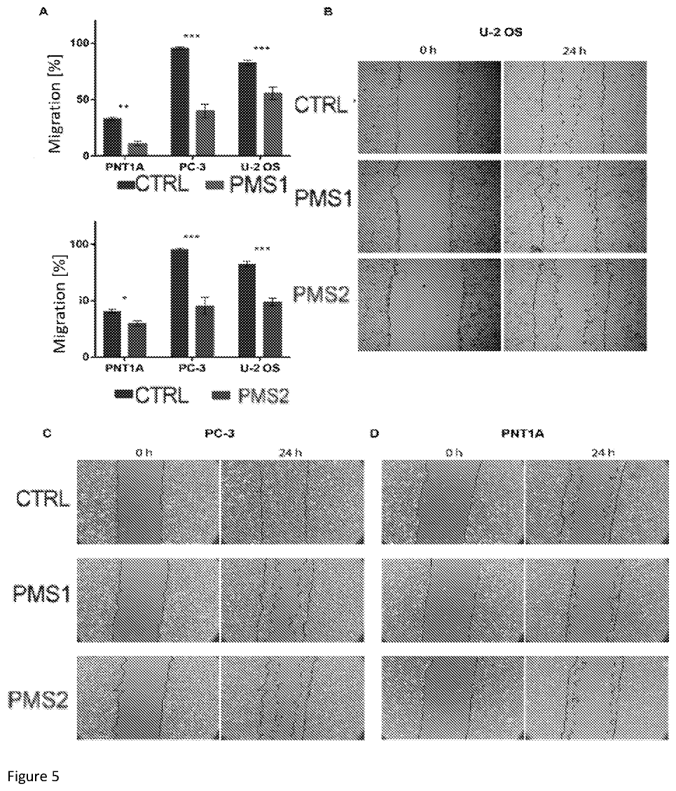

FIG. 6 shows the effect of substances 1 and 2 on cell migration and invasiveness. The figure depicts determination of the effect of substances 1 and 2 on cell migration and invasiveness. A) Normalized cell index for cell migration. B) Normalized cell index for cell invasiveness. C) Cell lines without and in the presence of tested substances. D) Median Young's modulus of elasticity.

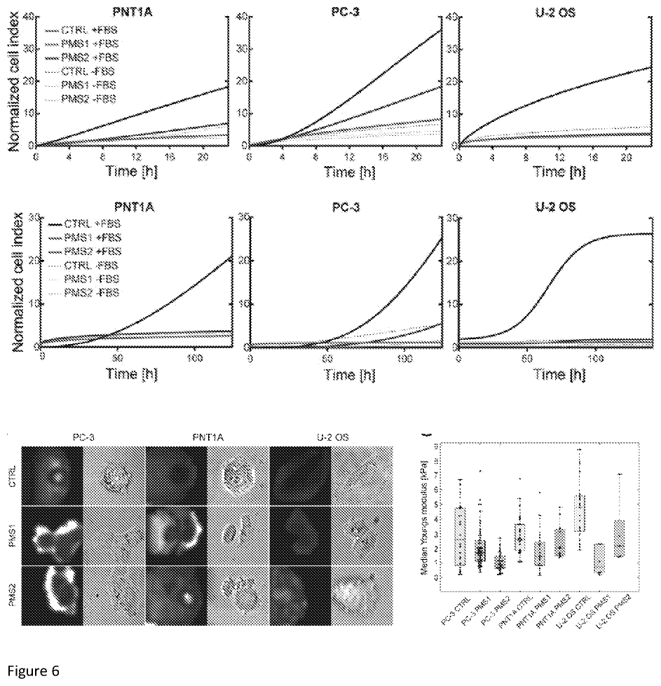

FIG. 7 shows the effect of substances 1 and 2 on the mitochondrial network and vitality. The figure depicts effect of substances 1 and 2 on mitochondrial activity and mitochondrial distribution. A) mitochondrial network (active mitochondria marked in green) U-2 OS without and in the presence of substances 1 and 2. B) Lipid peroxidation (marked in green) in U-2 OS without and in the presence of substances 1 and 2). C) Quantification of the effect of substances 1 and 2 on the U-2 OS mitochondrial network. d) Quantification of the effect of substance 1 and 2 on the PC-3 mitochondrial network.

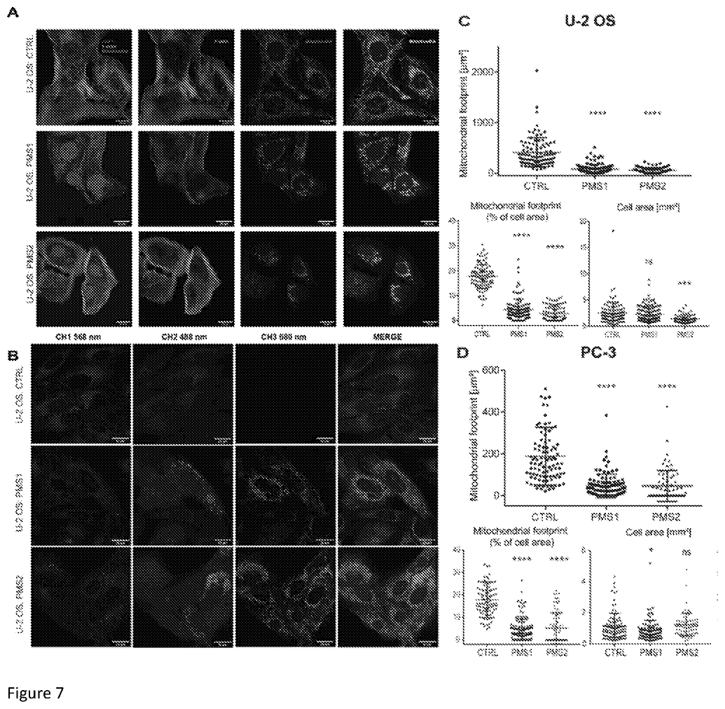

### EXAMPLES

### Example 1 Preparation of Substance 1 of General Formula I

The flask was charged with 2-(4-pyridyl) malondialdehyde (150 mg, 1.0 mmol), 2-methyl-3-propylbenzothiazolium iodide (640 mg, 2.01 mmol) and dry n-butanol (25 mL). The mixture was stirred for 18 hours at 110° C. After cooling to room temperature, the mixture was filtered. The solid was washed with ethanol (3×5 mL) and dried in vacuo. Product 1 was obtained as a green powder, 498 mg, 79%. 1H NMR (300 MHZ, DMSO-d6, 25° C.): 8.94 (2H, d, J=6.4 Hz), 8.14-7.80 (8H, m), 7.60 (2H, t, J=8.2 Hz), 7.46 (2H, t, J=7.6 Hz), 6.20 (2H, d, J=13.8 Hz), 4.28 (4H, bs), 1.71 (4H, sextet, J=7.0 Hz), 0.85 (6H, t, J=7.0) Hz). 13C NMR (126 MHZ, DMSO-d6, 25° C.): 165.9, 148.2, 143.6, 141.3, 128.2, 127.1, 125.5, 123.2, 114, 0, 98.3, 47.5, 20.9, 10.8. HRMS for C₃₀H₃₀N₃S₂, calcd: 496.1876 [M]⁺, found: 496.1879 [M]⁺. Elemental analysis for C₃₀ H₃₀ N₃ S₂ calcd: C, 57.78; H, 4.85; N, 6.74; found: C, 57.59; H, 4.93; N, 6.67.

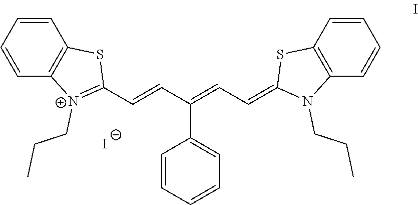 [SMILES: CCCN1/C(=C/C=C(/C=C/c2sc3ccccc3[n+]2CCC)c2ccccc2)Sc2ccccc21 ; [I-]]

### Example 2 Preparation of Substance 2 of General Formula I

The flask was charged with 2-(4-pyridyl) malondialdehyde (353 mg, 2.37 mmol), 2,3,3-trimethyl-1-propyl-3H-indol-1-ium iodide (1600 mg, 4.86 mmol), dry butanol (30 ml) and three drops of triethylamine. The mixture was stirred for 18 hours at 110° C. After this time, the mixture was cooled to room temperature and evaporated to dryness. The product is separated by column chromatography (silica gel, eluent: chloroform/methanol 10:1). The product was separated as a dark blue band. The separated fraction was evaporated to dryness and ethyl acetate was added to the residue and the whole mixture was sonicated for 2 minutes. The pure product was separated by filtration. The yield was 347 mg, 80% as a dark green powder. ¹H NMR (300 MHZ, DMSO-d6, 25° C.): 8.78 (2H, d, J=5.6 Hz), 8.50 (2H, d, J=14.3 Hz), 7, 67 (2H, d, J=7.4 Hz), 7.46-7.24 (8H, m), 5.59 (2H, d, J=14.4 Hz), 3.77 (4H, t, J=6.7 Hz), 1.75 (12H, s), 1.55 (4H, sextet, J=7.4 Hz), 0.74 (6H, t, J=7.3 Hz); ¹³C NMR (126 MHZ, DMSO-d6, 25° C.): 173.3, 151.7, 150.4, 144.0, 142.0, 141.2, 131.1, 128.5, 125, 3, 125.2, 122.6, 111.5, 100.7, 49.2, 45.0, 27.0, 20.2, 11.0; HRMS for C₃₆H₄₂N₃ calculated: 516.3373 [M]⁺, found: 516.3377 [M]⁺. Elemental analysis for C₃₆H₄₂IN₃ calcd: C, 67.18; H, 6.58; N, 6.53; found: C, 67.32; H, 6.51; N, 6.74;

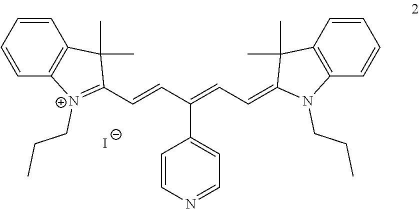 [SMILES: CCCN1/C(=C/C=C(/C=C/C2=[N+](CCC)c3ccccc3C2(C)C)c2ccncc2)C(C)(C)c2ccccc21 ; [I-]]

### Example 3 Preparation of Substance 3 of General Formula I

The flask was charged with 2,3,3-trimethyl-1-propyl-3H-indol-1-ium iodide (50 mg, 0.15 mmol), 2-(4-nitrophenyl)-4-(3-propylbenzo[d]thiazol-2 (3H)-ylidene) but-2-enal (50 mg, 0.14 mmol) and dry butanol (7 mL). The mixture was stirred for 18 hours at 110° C. After cooling to room temperature, the mixture was filtered. The solid was washed with butanol (5 mL) and dried in vacuo. Product 3 was obtained as a green powder, 44 mg. Another portion of the product was obtained by column chromatography of the evaporated filtrate (silica gel, eluent: dichloromethane/methanol 10:1). The product was separated as a dark blue band. The separated fraction was evaporated to dryness and diethyl ether (5 mL) was added to the residue and the whole mixture was sonicated for 2 min. The pure product was separated by filtration. An additional 12 mg of product was obtained. The total yield of compound 3 was 56 mg (54%) as a metallic bright green powder. 1H NMR (300 MHZ, DMSO-d6, 25° C.): 8.50-7.10 (16H, m), 6.01 (2H, d, J=13.9 Hz), 5.51 (2H, d, J=13.4 Hz), 4.27 (2H, bs), 3.70 (2H, bs), 1.71 (6H, s), 1.66 (2H, m), 1.53 (2H, m), 0.77 (6H, m); ¹³C NMR (126 MHZ, DMSO-d6, 25° C.): 170.5, 167.3, 151.7, 148.6, 146.7, 143.2, 142.4, 141.3, 140, 5, 131.6, 129.3, 128.5, 128.3, 126.2, 126.0, 124.2, 123.9, 123.5, 122.4, 114.7, 110.4, 101.5, 97.7, 48.3, 48.1, 44.3, 27.3, 21.1, 20.0, 11.1, 10.9; HRMS [M]⁺ (m/z) for C₃₄H₃₆N₃O₂S calcd: 550.2523, found: 550.2528.

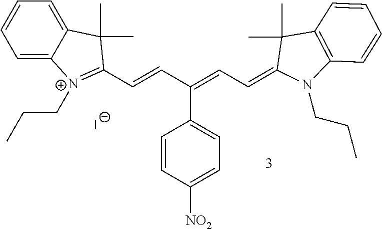 [SMILES: CCCN1/C(=C/C=C(/C=C/C2=[N+](CCC)c3ccccc3C2(C)C)c2ccc([N+](=O)[O-])cc2)C(C)(C)c2ccccc21 ; [I-]]

### Example 4 Preparation of Substance 4 of General Formula I

The flask was charged with 2,3,3-trimethyl-1-propyl-3H-indol-1-ium iodide (1320 mg, 4.01 mmol), malondialdehyde dianil hydrochloride (520 mg, 2.01 mmol) and dry pyridine (25 mL). The mixture was stirred for 18 hours at 90° C. After this time, the mixture was cooled to room temperature and evaporated to dryness. The product is separated by column chromatography (silica gel, eluent: dichloromethane/methanol 10:1). The product was separated as a dark blue band. The separated fraction was evaporated to dryness and diethyl ether (5 mL) was added to the residue and the whole mixture was sonicated for 2 min. The pure product was separated by filtration. 158 mg (74%) of compound 4 was obtained as a dark green powder. 1H NMR (300 MHZ, DMSO-d6, 25° C.): 8.34 (2H, t, J=12.9 Hz), 7.63 (2H, d, J=7.35 Hz), 7, 41 (4H, m), 7.24 (2H, m), 6.59 (1H, t, J=12.3 Hz), 6.32 (2H, d, J=13.8 Hz), 4, 08 (4H, t, J=7.0 Hz), 1.72 (4H, m), 1.69 (12H, s), 0.95 (6H, t, J=7.3 Hz); ¹³C NMR (126 MHz, DMSO-d6, 25° C.): 172.7, 154.0, 142.1, 141.1, 128.4, 125.5, 124.7, 122.4, 111, 2, 103.2, 48.9, 44.7, 27.2, 20.4, 11.0; HRMS [M]⁺ (m/z) for C₃₁H₃₉N₂ calcd: 439.3108, found: 439.3109.

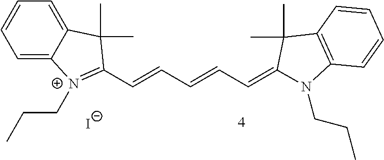 [SMILES: CCCN1/C(=C/C=C/C=C/C2=[N+](CCC)c3ccccc3C2(C)C)C(C)(C)c2ccccc21 ; [I-]]

### Example 5 Preparation of Other Substances of General Formula I

The substances listed in Table 1 were prepared according to the procedures described in Examples 1-3. Part of the prepared substances, including description of preparation, purification and characterization are part of our earlier patents and publications [Dyes Pigments 172 (2020) 107802; Dyes Pigments 107 (2014) 51-59; Bioorg. Chem. 82 (2019) 74-85; CZ304094B6; CZ304948B6; GB2567124; Bioconjugate Chem. 2013, 24, 1445-1454].

TABLE 1
Prepared substances

| Structure | |
|---|---|
| 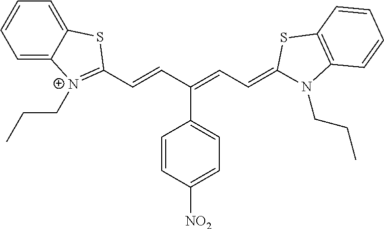 [SMILES: CCCN1/C(=C/C=C(/C=C/c2sc3ccccc3[n+]2CCC)c2ccc([N+](=O)[O-])cc2)Sc2ccccc21] | 5 |
| 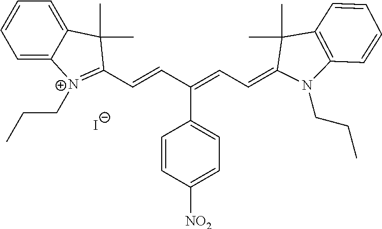 [SMILES: CCCN1/C(=C/C=C(/C=C/C2=[N+](CCC)c3ccccc3C2(C)C)c2ccc([N+](=O)[O-])cc2)C(C)(C)c2ccccc21 ; [I-]] | 6 |
| 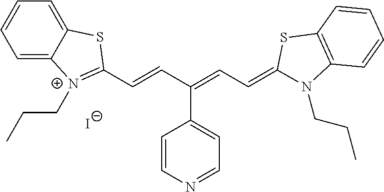 [SMILES: CCCN1/C(=C/C=C(/C=C/c2sc3ccccc3[n+]2CCC)c2ccncc2)Sc2ccccc21 ; [I-]] | 7 |
| 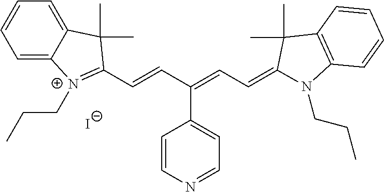 [SMILES: CCCN1/C(=C/C=C(/C=C/C2=[N+](CCC)c3ccccc3C2(C)C)c2ccncc2)C(C)(C)c2ccccc21 ; [I-]] | 8 |
| 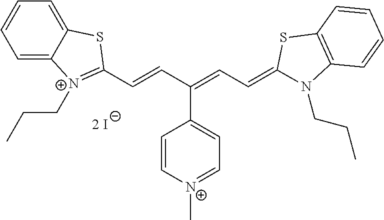 | 9 |
| 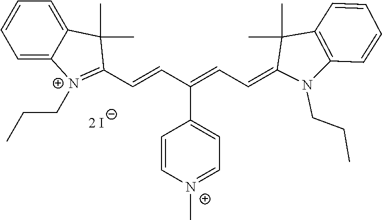 | 10 |
| 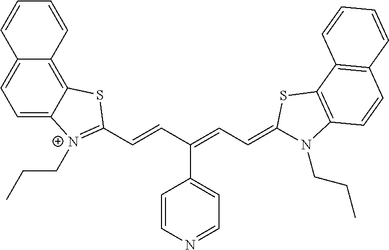 [SMILES: CCCN1/C(=C/C=C(/C=C/c2sc3c4ccccc4ccc3[n+]2CCC)c2ccncc2)Sc2c1ccc1ccccc21] | 11 |
| 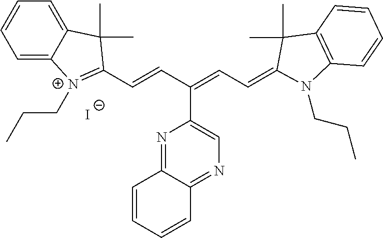 [SMILES: CCCN1/C(=C/C=C(/C=C/C2=[N+](CCC)c3ccccc3C2(C)C)c2cnc3ccccc3n2)C(C)(C)c2ccccc21 ; [I-]] | 12 |
| 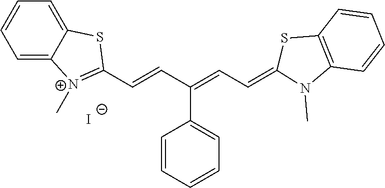 [SMILES: CN1/C(=C/C=C(/C=C/c2sc3ccccc3[n+]2C)c2ccccc2)Sc2ccccc21 ; [I-]] | 13 |
| 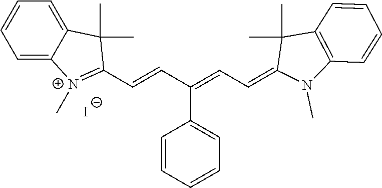 [SMILES: CN1/C(=C/C=C(/C=C/C2=[N+](C)c3ccccc3C2(C)C)c2ccccc2)C(C)(C)c2ccccc21 ; [I-]] | 14 |
| 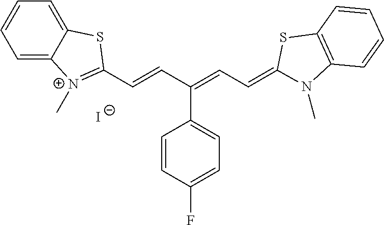 [SMILES: CN1/C(=C/C=C(/C=C/c2sc3ccccc3[n+]2C)c2ccc(F)cc2)Sc2ccccc21 ; [I-]] | 15 |
| 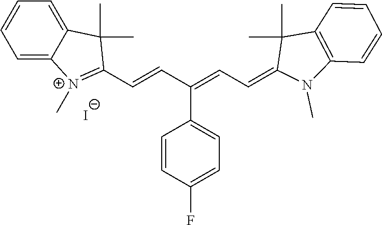 [SMILES: CN1/C(=C/C=C(/C=C/C2=[N+](C)c3ccccc3C2(C)C)c2ccc(F)cc2)C(C)(C)c2ccccc21 ; [I-]] | 16 |
| 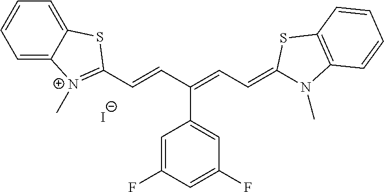 [SMILES: CN1/C(=C/C=C(/C=C/c2sc3ccccc3[n+]2C)c2cc(F)cc(F)c2)Sc2ccccc21 ; [I-]] | 17 |
| 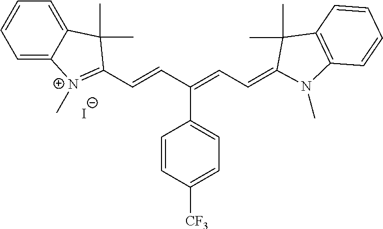 [SMILES: CN1/C(=C/C=C(/C=C/C2=[N+](C)c3ccccc3C2(C)C)c2ccc(C(F)(F)F)cc2)C(C)(C)c2ccccc21 ; [I-]] | 18 |
| 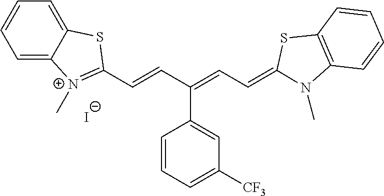 [SMILES: CN1/C(=C/C=C(/C=C/c2sc3ccccc3[n+]2C)c2cccc(C(F)(F)F)c2)Sc2ccccc21 ; [I-]] | 19 |
| 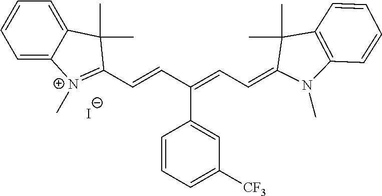 [SMILES: CN1/C(=C/C=C(/C=C/C2=[N+](C)c3ccccc3C2(C)C)c2cccc(C(F)(F)F)c2)C(C)(C)c2ccccc21 ; [I-]] | 20 |

### Example 6: Inhibition of DHODH-Dependent Respiration

DHODH-dependent respiration was performed as follows. Cells were trypsinized, washed with PBS, resuspended in an amount of 2×10⁶ cells per ml Mir05 medium (0.5 mM EGTA, 3 mM MgCl₂, 60 mM K-lactobionate, 20 mM taurine, 10 mM KH₂PO₄, 110 mM sucrose, 1 g/L bovine serum albumin, 20 mM Hepes, pH 7.1 at 30° C.) and transferred to the chamber of the Oxygraph-2k instrument (Oroboros). Respiration was measured at 37° C. Cells were permeabilized with 5 μg of digitonin per 10⁶ cells, and complex I inhibitor (0.3 μM rotenone) and test substance were added at concentrations from 1 to 4 nM before adding the substrate. DHODH-mediated respiration was assessed by subtracting the residual rate of respiration remaining after the addition of 30 μM leflunomide from the rate of respiration in the presence of 1 mM dihydroorotate (DHO), 3 mM ADP, and 10 μM cytochrome c.

### Example 7. Docking Study of Substances 1 and 2 with DHODH

Protein details: Dihydroorotate dehydrogenase (DHODH); RCSB PDB: crystal structure of 3U2O DHODH complexed with a small molecule inhibitor; Docking tools: Molegro Virtual Docker mvd 7.0.0 and Chimera 1.15.

The crystal structure model of the DHODH protein in complex with a small molecule inhibitor (3U2O) was downloaded from the RCSB PDB. Molegro Virtual Docker MVD 7.0.0 was used for docking with ligands. It uses the MolDock scoring system and is based on a hybrid search algorithm, the so-called directed differential evolution. This algorithm combines a differential evolution optimization technique with a cavity prediction algorithm. The crystal structure of the protein from the RCSB PDB was uploaded to the MVD 7.0 platform for the molecular docking process. It has a built-in cavity detection algorithm that identifies potential binding sites referred to as active sites/cavities. The Moldock S E search algorithm was used and the number of runs was 10, with a maximum number of iterations of 2000 for a population size of 50 and an energy threshold of 100. At each step, the smallest “min” torsions/translations/rotations were searched and the molecule with the lowest energy was preferred. After the molecular docking simulation, the obtained positions (binding modes) were classified according to the re-rank score.

Selected ligands were prepared manually using the Chimera V1.15 program and prepared using the built-in MVD program. Molecular docking was performed on amino acids found to be part of the 3U2O interaction with the largest cavity in which a known small molecule inhibitor binds. Grid resolution was set to 0.3 Å. Maximum iterations and maximum population size were set to 1500 and 50, resp. The resulting docking positions based on MolDock scores were imported and visualized and are shown in Table 1.

TABLE 1
Binding energies of tested substances for DHODH

| Ligand | Binding energy (kcal/mol) |
|---|---|
| 03U-1 (known inhibitor) | −148.2 |
| Substance 1 | −170.4 |
| Substance 2 | −141.5 |
| BAY-2402234 | −161.2 |

### Example 8. Cytotoxic Properties of Substances 1 and 2

The MTT assay was used to determine cell viability. After passage, the cell suspension in growth medium was diluted to a concentration of 2,000-10,000 cells/200 μl and transferred to a 96-well plate. A positive and negative control were placed on each plate. Plates were incubated for 2 days at 37° C. to ensure cell adhesion. Substance 1 and substance 2 were added to the fresh medium in increasing concentrations (0-10 μmol/L for both substances). Plates after addition of substances were incubated for 24 hours. Subsequently, the medium was changed to fresh medium with MTT (4:1, MTT 5 mg/ml in PBS) and incubated for 4 h in an incubator in the dark. DMSO was used to dissolve the MTT-formazan crystals and the absorbance was measured at 570 nm (VersaMax microplate reader, USA). IC₅₀ inhibitory concentrations were subsequently calculated and used in further experiments.

### Example 9. Migrastatic Properties of Substances 1 and 2

After passage, each cell line was resuspended and seeded in a 24-well plate, with the amount of cells per well in 500 μl medium optimized for each cell line. After 48 h, the cells were 100% confluent and a notch was made and 1 μM concentration of substance 1 or 2 was added. After gentle washing and media exchange, each well was photographed at time 0 and at 24 h in the same location. The photographs were analyzed and the software calculated the percentage of open groove area. Each cell line was analyzed in min. twenty four repetitions.

### Example 10. Invasiveness (Metastatic Potential In Vitro) and Determination of Cell Migration after Application of Substances 1 and 2

The xCELLigence system based on real-time cell impedance analysis (RTCA) was used to determine invasiveness and migration rates. The xCELLigence system consists of four main components: an RTCA DP station, an RTCA computer with integrated software, and CIM 16 disposable plates. First, the optimal cell seeding concentration for the proliferation and invasiveness assay was determined. After seeding the total number of cells in 200 μl medium into each well in E-plate 16, cell adherence and proliferation were monitored every 15 minutes. For the invasiveness assay, the optimal response was found in a well containing 20,000 cells. After coating the upper wells with matrigel and adding FBS as a chemoattractant, cells were seeded in 100 μl medium in each well of a CIM-plate 16. Adherence and growth of cells through the matrigel was monitored every 15 minutes. The duration of all experiments was 150 hours. The relative invasiveness rate was defined as the cellular index for matrigel-coated wells (cells must spread/pass through the matrigel to generate a signal) at a given time point. The impedance of electron flow due to adherent cells is given by a unitless parameter called the cell index (CI), where CI=(impedance at time point n-impedance in the absence of cells)/nominal impedance value). In order to compare individual cell lines/treatment, these cell indices were normalized to 1.0 at the time the test agents were added. Normalized cell indices are shown in Tables 2 and 3. Concentrations of compounds were chosen based on the IC₅₀ inhibitory concentration for each cell line.

TABLE 2
Effect of tested substances 1 and 2 on cell migration

| Normalized cell index | Control | Substance 1 | Substance 2 |
|---|---|---|---|
| PNTA | 18.6 | 3.4 | 7.0 |
| Control-substance | — | 15.2 | 11.6 |
| PC-3 | 38.8 | 8.8 | 19.5 |
| Control-substance | | 30.0 | 19.3 |
| U-2 OS | 24.3 | 3.5 | 3.9 |
| Control-substance | | 20.8 | 20.4 |

TABLE 3
Effect of tested substances 1 and 2 on cell invasiveness

| Normalized cell index | Control | Substance 1 | Substance 2 |
|---|---|---|---|
| PNTA | 19.7 | 2.8 | 3.2 |
| Control-substance | — | 16.9 | 16.5 |
| PC-3 | 22.8 | 1.4 | 5.4 |
| Control-substance | — | 21.4 | 17.4 |
| U-2 OS | 25.6 | 1.3 | 1.9 |
| Control-substance | — | 24.3 | 23.7 |

### Example 11. Intracellular Localization of Substances 1 and 2 and Effect on the Mitochondrial Network

Substance 1 or 2 was added to the tested lines (2×10⁶ cells per ml medium) so that its concentration was 200 nM. A Leica D M RXA microscope (equipped with a DMSTC motorized stage, a Piezzo z-motion, a MicroMax CCD camera, a CSU-10 confocal unit, and 488, 562, and 714 nm laser diodes with AOTF) (100× Plan Fluotar objective with by oil immersion, NA 1.3) was used to take detailed images of the cells. A total of 50 cuts were acquired with a Z step size of 0.3 μm. Subsequently, the image data was analyzed. Actin was labeled with Alexa Fluor™ 488 phalloidin (A12379, Invitrogen); 1 unit per slide. Duolink® In Situ Mounting Medium with DAPI (DUO82040) was used for mounting. Cells were fixed in 3.7% paraformaldehyde and permeabilized with 0.1% Triton X-100. The mitochondrial network was labeled with different types of MitoTracker@ probes.

### INDUSTRIAL APPLICABILITY

The invention can be used in the pharmaceutical industry for the preparation of new drugs that target the inhibition of dihydroorotate dehydrogenase.

1. A compound of general formula I 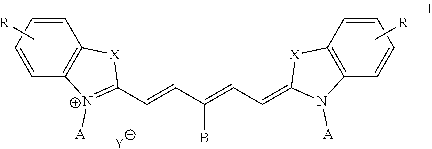
where both terminal heteroaromatic groups of the methinium chain are identical or different and are benzothiazole, naphthothiazole, benzimidazole, naphthoimidazole, benzooxazole, naphthooxazole, benzoselenazole, naphthoselenazole, quinoline, benzoquinoline, indole or benzoindole, the particular structure of which is characterized by the groups A, B, X, Y, with one or more R groups on both terminal heteroaromatic groups of the methinium salt, where R is H, C1 to C12 alkyl, glycol chains with 1 to 8 glycol (OCH₂CH₂) repeating units terminatimg with an O—C1 to C12 alkyl substituent or OH group, alkyl C1 to C8 sulfonic acids or their corresponding lithium, sodium or potassium salts, allyl, propargyl, phenyl, benzyl, pyridyl, halogen, CH₂OR′, OR′, CHF₂, CF₃, OCF, OCOR′, CN, CHO, COOR′, CONHR′, CONR′₂, CONHOR′, CONHNHR′, CONHNR′₂, N₃, NO₂, SR′, SCN, NHR′, NR′₂, NHCOR′, NHCONHR′, NHCONR′₂, NHCSNHR′, NHCSNR′₂, NHSO₂NHR′, NHSO₂NR′₂, NHCOOR′, N(COOR′)₂, B(OR′)₂, SO₃R′, SO₂NHR′, SO₂NR′₂, SO₂R′, where R′ is H, alkyl C1 to C12, phenyl, p-tolyl, benzyl, allyl, propargyl, CF₃; CH═CH—CH═CH (i.e. a fused benzene nucleus), where X is O, S, Se, CR′₂, NR′, CH═CH, where R′ has the above meaning; where A is alkyl C1 to C12, benzyl, allyl, propargyl, a glycol chain with a number of 1 to 8 glycolic (CH₂CH₂O) repeating units terminating with the substituent R′, (CH₂)ⱼCOR′, (CH₂)ⱼCOOR′, (CH₂)ⱼSO₃R′, (CH₂)ⱼSO₃H, (CH₂)ⱼCONHR′, (CH₂)ⱼCONR′₂, where j is in the range of 1 to 12 and R′ has the above meaning; where B is phenyl, pyridyl, pyrazinyl, quinolyl, quinoxalyl, furanyl, thienyl, benzoxazolyl, benzothiazolyl, which may be further substituted by one or more of the same or different substituents R, where R has the above meaning, where in the case of doubly charged salts the group B is formed by pyridyl or quinolyl quaternized on its nitrogen atom by A, where A is as defined above, where Y is acetate, acetylacetate, adipate, ascorbate, benzoate, besylate, borate, bromide, butanoate, citrate, deoxycholate, dihydrogen phosphate, phenylacetate, fluoride, phosphate, fumarate, gallate, glutarate, hexafluorophosphate, hippurate, hydrogen sulfate, chloride, perchlorate, cholate, isocyanate, isonicotinate, iodide, caprylate, cyanate, lactate, laurate, lithocholate, malate, maleate, malonate, mandelate, mesylate, monohydrogen phosphate, formate, myristate, napsylate, nicotinate, nitrate, nonafluorobutylsulfonate, oleate, oxalate, oxopropanoate, palmitate, picrate, pimelate, propionate, rhodanide, salicylate, sebacate, cinnamate, stearate, suberate, succinate, sulfate, tetrafluoroborate, tosylate, triflate, trifluoroacetate, trichloroacetate, carbonate, valerate, tartrate, and salts of natural amino acids,
for the use as an inhibitor of dihydroorotate dehydrogenase.

2. The compound of general formula I, wherein the symbols A, B, X and Y have the meaning as defined in claim 1, for use as an antimetastatic agent.

3. The compound of general formula I, wherein A, B, X and Y have the meaning as defined in claim 1, for use in the treatment of metastases of solid tumors.

4. Use of the compound of the general formula I, where the symbols A, B, X and Y have the meaning as defined in claim 1, for the production of a medicament for the treatment of metastatic oncological diseases.
